(19) United States
(12) Patent Application Publication
(10) Pub. No.: US 2023/0346614 A1
(43) Pub. Date: Nov. 2, 2023

(54) NIPPLE ABSORBENT ELEMENT

(71) Applicants: Anna FORLESI, Cesena (Forlì-Cesena) (IT); Camilla Giulia BAZZANTI, Firenze (IT)

(72) Inventors: Anna FORLESI, Cesena (Forlì-Cesena) (IT); Camilla Giulia BAZZANTI, Firenze (IT)

(21) Appl. No.: 17/903,235

(22) Filed: Sep. 6, 2022

(30) Foreign Application Priority Data
Apr. 27, 2022 (IT) 202022000001682

(51) Int. Cl. A61F 13/539; A61F 13/14

(52) U.S. Cl. CPC A61F 13/539; A61F 13/141; A61F 2013/15016

(57) ABSTRACT

Nipple absorbent element, including a body having a substantially flat development and having an inner surface configured to be in contact with the nipple and an outer surface; wherein at least said inner surface is made of soft, yieldable and absorbent material to retain liquids exiting the nipple; and wherein the outer surface has a fluorescent graphic representation corresponding to a respective condition of the breast for feeding.

This application claims priority to Italian Patent Application 202022000001682 filed Apr. 27, 2022, the entirety of which is incorporated by reference herein.

The present invention refers to a nipple absorbent element, in particular used during the breastfeeding period.

As known, in order to absorb milk and/or colostrum spillage that frequently occurs in the breastfeeding phases, natural fabric elements are used directly applied on the nipples and retained thereon by the garments, typically by the bra.

These elements are also intended to protect the nipple surface, which is particularly sensitive during the breastfeeding period.

In particular, these elements are made of soft, absorbent material, such as cotton, which is suitable for the contact with the nipple and at the same time suitable to be washed for reuse thereof.

The elements have a substantially flat shape and in use are counter-shaped to the respective breast so as to assume a spherical cap profile. These elements therefore define an inner surface that in use has a concave development, which can abut to the skin for the comfortable containment of the nipple, and an outer surface in contact with the garments (bra).

The elements thus produced are removed during breastfeeding for each individual breast and repositioned between the nipple and the garment at the end of the feeding. In this way, subsequent spillages of liquid can be absorbed by the element avoiding wetting the bra or the garments in contact with the breast.

The absorbent elements described above, although structurally simple and practical to use, have some limitations of use.

Indeed, a first major drawback stems from the difficulty on the part of the mother in quickly identifying the breast most ready for feeding.

In fact, it is not always immediate for the mother to recognise, e.g. by manual pressure, the breast that is richest in milk and therefore most suitable to start a new feeding.

In this context, in order to homogeneously alternate the feedings between the two breasts, it is difficult to recognise the breast that has just provided milk from the one ready for suckling.

This drawback is even more relevant in situations of night feeding where the visual perception of the mother is altered by the absence of light. In this case, in fact, it is difficult to perceive the largest breast and with the greatest production of milk that is therefore ready for feeding.

In this context, the technical task underlying the present invention is to propose a nipple absorbent element that is free from the drawbacks of the prior art mentioned above.

In particular, aim of the present invention is to propose a nipple absorbent element able to signal which of the two breasts has provided the last feeding and consequently which is ready for a new feeding.

A further aim of the present invention is to make available a nipple absorbent element that is structurally simple, inexpensive and easy to use.

The specified technical task and aims are substantially achieved by a nipple absorbent element as described in one or more of the accompanying claims.

Further features and advantages of the present invention will become clearer from the indicative, and therefore non-limiting, description of a preferred but non-exclusive embodiment of a nipple absorbent element.

Such description will be set forth herein below with reference to the accompanying drawings, provided for merely indicative and therefore non-limiting purposes, wherein:

FIG. 1 shows a perspective view of a nipple absorbent element according to the present invention;

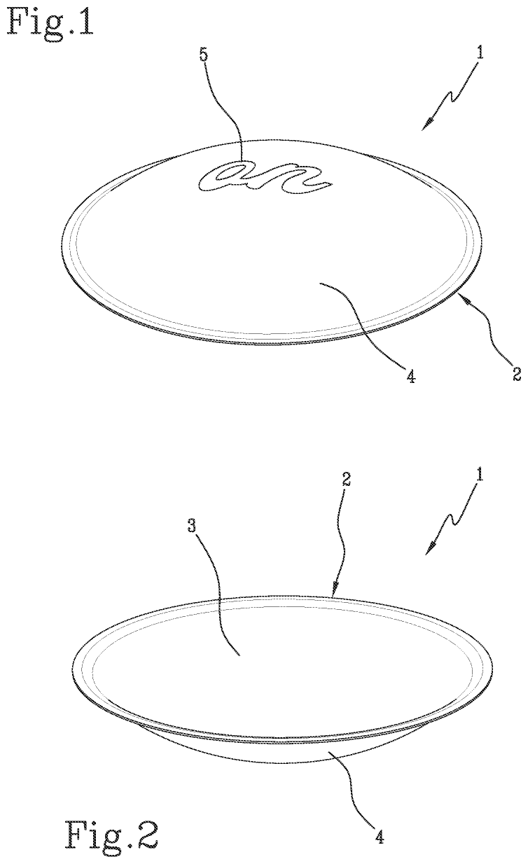

FIG. 2 shows a perspective view of an inner area of the element of FIG. 1; and

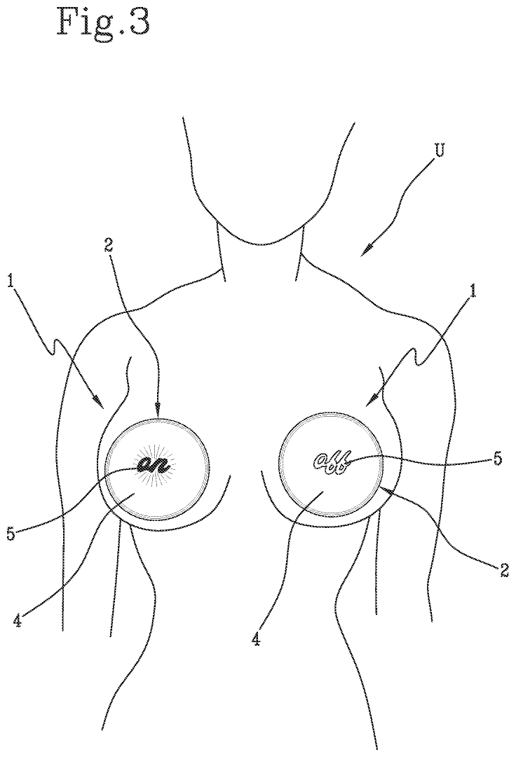

FIG. 3 shows a front view of two absorbent elements according to the present invention and in a respective condition of use.

With reference to the accompanying figures, reference numeral 1 globally denotes a nipple absorbent element.

The element 1 comprises a flat body 2 with circular peripheral development. In addition, the body 2 is made of soft material in order to adapt to the convexity of the breast at the nipple. In this way, the body 2 in use, i.e. applied to the breast, has a slightly spherical cap shape as illustrated by way of example in FIGS. 1 and 2.

In greater detail, the body 2 has an inner surface 3 configured to be in contact with the nipple of the user “U”.

Advantageously, the inner surface 3 is made of soft, yieldable and absorbent material, such as a vegetable fibre fabric. In this way, the inner surface 3 can be adapted to the profile of the nipple, giving a comfortable feeling in the contact with the skin. Furthermore, the nature of the material with which the inner surface 3 is made allows the absorption and retention of liquid milk exiting the nipple between one feeding and the other (milk or colostrum).

Preferably, the material of the inner surface 3 is at least in part of cotton and/or at least in part made of vegetable fibre, preferably bamboo.

The body 2 further comprises an outer surface 4 opposite the inner surface 3 and configured to remain visible. In use, the outer surface 4 is convex and also configured to be abutted to the garment, typically the bra, in order to keep the body 2 in the position abutted to the nipple.

In accordance with a main aspect of the present invention, the outer surface 4 has a fluorescent graphic representation 5 corresponding to a respective condition of use or non-use of the breast for feeding.

In other words, the graphic representation 5, which appears to be visible both with light and in the absence of light thanks to its fluorescent characteristic, gives the user “U” a visual indication of the breast that previously provided the feeding and of the breast ready for a new feeding.

In the accompanying figures, a graphic representation 5 is illustrated only by way of example and therefore not limiting, showing the letterings “on” and “off” in fluorescence.

In this way, the user immediately perceives, even in the dark, that she has to provide the feeding with the breast on which the “on” indication is placed.

In this context, it is important to use two elements 1 that differ between them in the graphic representation 5.

The fluorescent graphic representation 5 can advantageously be a design and/or a lettering and may be provided with any suitable colouring to be immediately visible, even in the absence of light.

Furthermore, the fluorescent graphic representation 5 may be made of a material activatable at a predetermined humidity value.

In this situation, the possible spillage of milk from the breast that is absorbed by the body 2 can increase the humidity of the body 2 itself to activate and make the representation 5 luminescent, thus warning the user “U” of the presence of milk for the subsequent feeding.

Preferably, the outer surface 4 is made of waterproof material, such as in particular thermoplastic polyurethane (TPU).

The present invention further refers to a protection element 1, intended for any part of the body and for protection, for example of a laceration or wound.

In this case, the inner surface 3 is configured to be in contact with the skin of a user at a respective area of the body. Also in this case, the outer visible surface 4 has a fluorescent graphic representation 5 identifying said area of the body.

For example, in this case the element 1 can consist of a patch adapted to indicate even in the dark the position of the wound that is covered by the patch itself. In this situation, the inner surface 3 has an adhesive layer to adhere to the user's skin.

Again, the fluorescent graphic representation 5 may be indicative of a date, corresponding to the day of application of the element 1 on the user's body, to provide the same user with a time indication on when to replace the element 1 itself.

The invention described above therefore brings important advantages and solves the drawbacks of the prior art. In fact, it should be noted that the element 1 is able to provide a visual indication adapted to signal which of the two breasts has provided the last feeding and which is instead ready for a new feeding.

It should also be noted that thanks to the fluorescent nature 5 of the graphic representation, it is possible to provide such a visual indication at any time, even in the dark, and therefore during night feedings.

Advantageously, the nipple absorbent element 1 is structurally simple, easy to use, washable and able to provide comfort given the nature of the material in contact with the nipple.

Again, the invention can also be used as a patch or in general as any skin protection system capable of providing visual indications to the user.

In this context, the element 1 appears to be extremely versatile precisely because it can be used for different purposes and in any area of the body.

1. Nipple absorbent element, comprising a body having a substantially flat development and having an inner surface configured to be in contact with the nipple and an outer surface; wherein at least said inner surface is made of soft, yieldable and absorbent material to retain liquids exiting the nipple; and wherein the outer surface has a fluorescent graphic representation corresponding to a respective condition of the breast for feeding.

2. Element according to claim 1, wherein the outer surface is made of waterproof material, preferably thermoplastic polyurethane.

3. Element according to claim 1, wherein the fluorescent graphic representation is a design and/or a lettering indicative of the breast that must or must not provide the feeding.

4. Element according to claim 1, wherein said fluorescent graphic representation is made of a material activatable at a predetermined humidity value.

5. Element according to claim 1, wherein said inner surface is at least in part made of vegetable fibre, preferably bamboo.

6. Protective element comprising an inner surface configured to be in contact with the skin of a user at a respective area of the body, and a visible outer surface; wherein the outer surface has a fluorescent graphic representation identifying said area of the body.

7. Element according to claim 6, wherein said inner surface has an adhesive layer for adhering to the skin of the user.

8. Element according to claim 6, wherein the fluorescent graphic representation is a design and/or a lettering.
